(19) United States
(12) Patent Application Publication
(10) Pub. No.: US 2003/0118631 A1
(43) Pub. Date: Jun. 26, 2003

(54) Anti-microbial soluble hemostatic gauze and method of making same

(76) Inventors: Gao Jing Xing, Xuanwu District (CN); Hong Zhang Xu, Xuanwu District (CN)

Correspondence Address: BUCHANAN INGERSOLL, P.C., ONE OXFORD CENTRE, 301 GRANT STREET, 20TH FLOOR, PITTSBURGH, PA, 15219, US

(21) Appl. No.: 10/028,230

(22) Filed: Dec. 21, 2001

(51) Int. Cl. A61K 38/14; A61K 31/7072; A61K 31/704; A61K 31/337; B32B 27/12; A61L 15/16

(52) U.S. Cl. 424/446; 442/123; 514/8; 514/449; 514/29; 514/39; 514/49

(57) ABSTRACT

A soluble hemostatic gauze is made from a cotton gauze immersed in an aqueous solution of from 90% to 95% ethanol at room temperature for at least 50 minutes and then exposed to an aqueous solution of NaOH at a temperature of from 35° to 40° C. for from 90 to 150 minutes. Next the cotton gauze is kept in an aqueous solution of acetic chloride at a temperature of from 35° to 40° C. for from 90 to 150 minutes and then washed in an aqueous solution of ethanol for a sufficient time to neutralize the cotton gauze and remove residue salts from the gauze resulting in a soluble hemostatic gauze. That soluble hemostatic gauze is then dried. An antibiotic or other pharmaceutically effective agent may be linked to the gauze molecules.

### FIELD OF INVENTION

The invention relates to a method for making hemostatic gauze that will dissolve after being applied to a wound or incision and clotting has occurred.

### BACKGROUND OF THE INVENTION

The control of bleeding is a serious problem in certain surgical procedures and in various types of emergency wounds. Bleeding from the kidney, brain or liver or the persistent oozing from severed capillaries and veins, for example, is particularly difficult to control by conventional means such as suturing or ligature, and in many cases, is serious enough to endanger life. Surgical hemostats consisting of conventional gauze pads or similar articles impregnated with a hemostatic material such as ferric chloride, thrombin or the like have been used for many years to arrest bleeding. Hemostats of this type cannot be left in a closed wound because foreign body tissue reaction would result.

Absorbable hemostatic materials have been developed which may be left in a wound site, if necessary, to control bleeding and will be eventually absorbed by the body without adverse tissue reaction. Such absorbable materials include the polymers and copolymers of lactide and glycolide, and oxidized cellulose. One method of preparation and use of oxidized cellulose as an absorbable hemostat is disclosed in U.S. Pat. No. 3,364,200. In that process oxidized cellulose is prepared by treating bright rayon regenerated cellulose with an oxidizing agent such as dinitrogen tetroxide in a Freon medium. After oxidation, the fabric is thoroughly washed with a solvent such as carbon tetrachloride followed by aqueous solution of 50 percent isopropyl alcohol and finally, with 99 percent isopropyl alcohol. Prior to oxidation, the hemostat is constructed in the desired form such as a gauze, knit, woven fabric, felt or integrated mass of staple fibers. Saferstein et al. in U.S. Pat. No. 5,134,229 disclose a process in which the neutralization of oxidized cellulose cloth is accomplished with mild neutralizing agents such as the sodium or potassium salts of weak acids, such as sodium acetate in water or in a mixture of water and alcohol. They report that the neutralized oxidized cellulose product produced by this process provides a strong (i.e. good tensile strength and integrity), convenient-to-use, storage-stable, and hemostatically effective cloth composition. The reference warns against use of stronger bases such as sodium hydroxide, ammonium hydroxide, sodium carbonate or sodium bicarbonate because such use was found to undesirably cause the neutralized cloth to unacceptably weaken and partially gel. Furthermore, they say that cellulose should be neutralized without chloride to avoid forming hydochloric acid that is a by-product of the reaction of chloride with the oxidized cellulose. Strong acids such as hydrochloric acid can cause oxidized cellulose to decompose into low molecular weight polymer, with loss of tensile strength and shortened shelf life. Weak acid does substantially no damage to the oxidized cellulose cloth. Saferstein et al. report that the use of strongly basic aqueous sodium hydroxide, ammonium hydroxide, and sodium carbonate solutions to neutralize oxidized cellulose cloth all lead to considerable shrinkage and loss of tensile strength to the cloth. Saferstein et al. also disclose that the oxidized cellulose material can be air-dried and impregnated with an effective amount of an acid-sensitive medicament, biologic or enzyme. For hemostasis, the medicament is preferably thrombin. For prevention of post surgical adhesions, the medicament is an adhesion-preventive substance, such as tissue plasminogen activator (t-PA), tissue plasminogen activator analogue (t-PAA), streptokinase, heparin, low molecular weight heparin, or pentasaccharide. Several other patents teach impregnation of a cellulose pad with an antibiotic or with another chemical that has some therapeutic benefit such as thrombin. However, they teach that the material is impregnated by dissolving the material in a solvent, immersing the cellulose in the solution and then freezing and drying the cellulose containing the solution. Examples of this teaching are in U.S. Pat. Nos. 2,517,772 to Doub, 4,453,410 to Cruz et al, 4,405,324 and 4,148,664 to Cruz, 5,484,913 to Stilwell and 4,404,970 to Sawyer. While this method of impregnation will place the antibiotic in the gauze, that antibiotic or other impregnated material will leach out if the gauze becomes wet. Consequently, there is a need for a hemostatic gauze containing an antibiotic that will not release the antibiotic when the gauze is exposed to water but will release the antibiotic when applied to a wound.

U.S. Pat. No. 3,666,750 discloses a hemostatic material formed from an oxidized cellulosic material. The patent teaches that cellulosic material including wood pulp and cotton is prepared by selective oxidation using nitrogen dioxide. Then the oxidized cellulosic material is treated with a borohydride, such as ammonium borohydride. Thereafter, the material is treated with a dilute acid to destroy the borohydride and washed in alcohol to remove any acid. The patent further teaches that the material be treated with a mixture of water and 20 to 85 percent methanol, ethanol, isopropanol or other alcohol before being exposed to the borohydride.

U.S. Pat. Nos. 4,453,410 and 4,405,324 to Cruz et al. teach that a cellulose structure can be treated with an alcohol followed by a 20% NaOH solution and then with a solution of 8 grams chloroacetic acid in 100% isopropanol to obtain an insoluble carboxymethyl cellulose. Later at column 9, lines 42-45, the '410 patent says that ring oxidation converts selectively the hydroxyl groups at the 2, 3 and 6 positions of the anhydroglucose unit into carboxyl groups. Cruz does not recognize an advantage to the presence of the carboxyl groups. In example 9 he suggests that there is a disadvantage to having them present. Furthermore, the resulting product is insoluble.

Although the benefits and usage of soluble hemostatic gauzes are well known, the known processes for making oxidized cellulose hemostatic gauze have several shortcomings. Consequently, there is a need for an inexpensive method of producing soluble hemostatic gauze, particularly a soluble hemostatic gauze containing an antibiotic or other pharmaceutically effective agent that will not leach out when the gauze is exposed to water.

### SUMMARY OF THE INVENTION

We provide a method for manufacturing soluble hemostatic gauze from cotton. The cotton is first woven into a gauze and degreased to provide a medical grade absorbent gauze. If desired one could obtain cotton gauze or medical grade absorbent gauze from a supplier and then process the gauze in accordance with the processing steps here described. In the present preferred method the medical grade absorbent gauze is first processed in ethanol, methanol or acetone at room temperature for 1 hour. Then the gauze is placed in an aqueous solution of from 90% to 95% of an alkaline material such as NaOH at 37° C. for 1 to 4 hours. Next the gauze is placed in a 20% to 80% solution of an acid or acid salt having a COOH group such as acetic chloride at 20° C. to 80° C. for 1 to 6 hours to attach COOH groups to the gauze molecules. After this treatment the gauze is washed in an ethanol, methanol or acetone at a temperature of from 10° C. to 60° C. for a sufficient time to neutralize the cotton gauze and remove residue salts from the gauze resulting in a soluble hemostatic gauze. This wash may be done in several steps. The gauze can be spun to remove the ethanol, methanol or acetone and dried in an oven at a temperature of from 20° C. to 120° C. At this point the gauze is a soluble hemostatic gauze material. Typically this material would be cut to size and packaged. Then the packaged material would be sterilized.

We prefer to soak the gauze in a solution of from 50% to 95% ethanol, methanol or acetone, 2% to 40% dimethyl sulfoxide and 2 mg/ml to 2 g/ml of an antibiotic or other pharmaceutically effective agent containing an amino-group to link the amino group onto a COOH group attached to molecules in the soluble hemostatic gauze. This step is performed between washing steps prior to drying. Suitable antibiotics include actinomycin, amphotericin B, chlindamycin, erythromycin, lincomycin and neomycin. The gauze may be impregnated with thrombin, tissue plasminogen activator, tissue plasminogen activator analogue, streptokinase, heparin, low molecular weight heparin, or pentasaccharide using known techniques.

### DESCRIPTION OF THE PREFERRED EMBODIMENTS

Our soluble hemostatic gauze is preferably made from cotton that has been treated with solutions of ethanol, sodium hydroxide and acetic chloride, also know as acetyl chloride. The cotton should be a medical grade absorbent gauze. Such a medical grade absorbent gauze can be obtained from a supplier or created from readily available cotton gauze. To create a medical grade absorbent gauze from cotton gauze, the cotton gauze is subjected to a degreasing process that is well know in the art. This process involves exposing the gauze to an aqueous solution of 75% to 80% ethanol, methanol or acetone. Although we prefer to use medical grade cotton gauze, any kind of cotton, silk, paper, hemp cloth or fiber could be substituted for the cotton gauze.

The medical grade absorbent gauze is first pre-washed in a 90% to 95% solution of ethanol, methanol or acetone at room temperature for 15 minutes to one hour. This step takes away all unnecessary particles and residue grease or oil that may be present. Next the gauze is placed in an aqueous solution of sodium hydroxide or other strong alkaline to break down the cotton fibers into small molecules and add OH⁻ bases onto these molecules. This exposure should be done at a temperature of from 20° C. to 50° C. for from 1 to 4 hours. We prefer to conduct this step at 37° C. for two hours. We prefer to use a concentration of 10% to 70% sodium hydroxide in ethanol solution having a concentration of from 50% to 95%. Other alkaline materials that could be used in place of sodium hydroxide are potassium hydroxide, calcium hydroxide and magnesium hydroxide, lithium hydroxide, soda ash (Na₂CO₃), and K₂CO₃. Next the gauze is treated in a solution of acetic chloride at a concentration of from 20% to 80% in an ethanol solution having a concentration of from 50% to 95%. This step is performed for 1 to 6 hours at a temperature of from 20° C. to 80° C. Any acetic acid, acetic acid salt, or any acid or acid salt with a COOH group/base in the molecule could be used in place of acetic chloride. This step adds COOH base onto the molecules in the gauze and completes the final reaction required to form the carboxy-methyl-cellulose molecule, thereby transforming the cotton gauze into a soluble hemostatic wound dressing. During this step alkaline salts are produced in the gauze and the resulting gauze has a pH above 7. At least one ethanol wash is used to remove the salts and neutralize the alkaline gauze. Indeed, we prefer to use three ethanol washes all at room temperature for respective periods of 30 minutes, 1 hour and 12 hours. However, the first and second washes could be at a temperature of from 10° C. to 60° C. for 10 to 60 minutes and the last wash may be within the same temperature range for from 1 to 12 hours. The gauze must be dried, cut to size and packaged. We prefer to dry the gauze in an oven at a temperature ranging from 40° C. to 120° C. The ethanol and water in the gauze evaporate in the oven to provide a soluble, white, soft and flexible gauze like material. The gauze could be spun immediately after the last wash and then allowed to air dry for at least 1 hour. After the gauze has been dried, cut and packaged the gauze is sterilized, preferably using irradiation.

The gauze can be sold and used in any convenient size. We prefer to provide rectangular and square gauze pads in sizes of 1.1×1.1, 5×5, 5×10, 5×20 and 10×20 centimeters. We also prefer to offer the gauze on adhesive strips. Like other medical grade gauze products our hemostatic gauze material can be easily cut with a knife or scissors.

Because the gauze molecules have a carboxyl group those antibiotics and other pharmaceutically effective compounds that have an amino group to link with the COOH group on the molecules will bond with those molecules when introduced into the gauze. Being so linked, the antibiotic will not leach from the gauze but remain attached to the cellulose molecule even after the gauze dissolves into the wound. Even though the antibiotic continues to have a carboxyl-methyl-cellulose molecule attached, the antibiotic still is effective. We attribute this result to the position of the attachment. For that reason, such a gauze is an effective product for treating wounds to prevent or stop infection. To link the antibiotic to the CMC molecule of the gauze, we prefer to soak the gauze in a solution of from 50% to 95% ethanol, methanol or acetone, 2% to 40% dimethyl sulfoxide (DMSO) and 2 mg/ml to 2 g/ml of the antibiotic. This step can be performed at temperatures of from 15° C. to 60° C. for from 0.1 hours to 4 hours. We prefer to soak the gauze for 0.5 hours at a temperature of 40° C. to link the antibiotic to the CMC molecule of the gauze. During soaking the amino-groups in the antibiotic will link onto a COOH group on the gauze molecules. This step is preferably performed before the final washing steps so that the washing steps will wash away any free antibiotics that are not linked to the gauze molecules. Suitable antibiotics include actinomycin, amphotericin B, chlindamycin, erythromycin, lincomycin and neomycin.

In addition to antibiotics, other pharmaceutically effective agents that have an amino group available to be linked to the carboxyl group could be used. Such agents include certain chemotherapeutic agents including paclitaxel, bleomycin, mitomycine, gemcitabine, and fluorouracil, or any chemical compound with an amino group in its molecular structure. They can be introduced into the gauze in a similar manner to the antibiotic and will attach to the cellulose molecules. Like the antibiotics, these materials will retain their effectiveness after the gauze dissolves.

If desired, the gauze can be impregnated with other antibiotics or other medicaments using conventional techniques. Those materials can be impregnated after the last wash step and prior to drying. Such other medicaments could be thrombin, tissue plasminogen activator, tissue plasminogen activator analogue, streptokinase, heparin, low molecular weight heparin, or pentasaccharide.

To measure the effectiveness of the our antimicrobial gauze, we tested an antibiotic containing soluble hemostatic gauze material made in accordance with the present invention against the same antibiotic alone. We prepared a growth base for anaeroboic bacteria by mixing 20 grams glucose, 5 grams NaCl, 10 grams protein base, 5 grams yeast powder, 3 grams soluble starch, 20 grams agar and 0.4 grams amino acid. These materials were dissolved in 150 ml distilled water to form a liver paste solution. Then we added 1000 ml. water, adjusted the Ph to 7.2 and autoclaved the material. We prepared another growth base for aerobic bacteria by combining 10 grams beef paste, 10 grams protein base, 3 grams soluble starch, 5 grams NaCl and 20 grams agar in distilled water. Then we added 1000 ml. water, adjusted the Ph to 7.2 and autoclaved the material. We also prepared a series of solutions of the antibiotic chlindamycin in a Ph7.8 phospharese solution. The solutions were: 1280 mg antibiotic/L, 640 mg/L, 320 mg/l, 160 mg/L, 80 mg/L, 40 mg/L, 20 mg/l, 10 mg/L, 5 mg/L, 2.5 mg/L and 1.25 mg/L. We then added 2 ml of each of the diluted antibiotic solutions into a petri dish. Next we added 18 ml of warm (50° C.) autoclaved bacteria growth base prepared as described above for the type of bacteria (anaerobic or aerobic) to be tested to petri dishes to make growth plates. At that point we had growth plates with final antibiotic concentrations of 128, 64, 32, 16, 8, 4, 2, 1, 0.5, 0.25, and 0.125 mg antibiotic per liter.

We prepared a second series of growth bases with anti-microbial soluble hemostatic gauze to which the antibiotic chlindamycin had been linked. The gauze was prepared in accordance with the following steps. First we dissolved 500 mg antibiotic in 100 ml 5% DMSO solution, and then we immersed 4 grams soluble hemostatic gauze into the solution, at 40° C. for 30 minutes. Next we washed the processed soluble hemostatic gauze with 400 ml ethanol solution and dried the processed anti-microbial soluble hemostatic gauze. We cut and weighed samples of the antimicrobial gauze to form a series of samples weighing 0.554 g, 0.276 g, 0.138 g, 0.068 g, 0.034 g, 0.0172 g, 0.0086 g, 0.0043 g, 0.00215 g, and 0.00107 g anti-microbial hemostatic gauze. We placed each sample into a flask, added 20 ml warm (50° C.) autoclaved bacteria growth base prepared as described above for the type of bacteria (anaerobic or aerobic) to be tested to the flask, and completely dissolved the soluble gauze. Then we poured the contents of each flask into a petri dish. This resulted in a series of test specimens having a final concentration of 128, 64, 32, 16, 8, 4, 2, 1, 0.5, 0.25, or 0.125 mg. of antibiotic per liter. We also prepared a series of controls by pouring 20 ml warm (50° C.) autoclaved bacteria growth bases prepared as described above for the two types of bacteria (anaerobic or aerobic) to be tested into the petri-dishes.

We inoculated all of the test specimens with one of several types of bacteria which are listed in Tables 1 through 4. We diluted each bacteria culture with Ph7.8 phosphorese solution to the concentration of 10⁸ cells/ml. Then we inoculated each plate with 0.5 ml of the specific bacteria solution and marked the plate. We put the plates inoculated with anaerobic bacteria into a 37° C. oven saturated with nitrogen for 48 hours with one non-inoculated growth plate as a negative control. We put the plates inoculated with aerobic bacteria into a regular 37° C. oven for 48 hours with one non-inoculated growth plate as negative control.

When the plates were removed from the oven we identified those plates which exhibited the minimum inhibition concentration for each bacteria and recorded the results. In Tables 1 through 4 we present the results for each type of bacteria tested. The tables report the lowest concentration of antibiotic and the lowest concentration of anti-microbial gauze that produced a minimum inhibition concentration. Those samples with concentrations higher than that listed in the tables also produced the same or better bacteria inhibition. For nearly every bacteria tested the minimum inhibition concentration of the antibiotic alone was the same as the minimum inhibition concentration for the antimicrobial gauze. Those results demonstrate that the antibiotic retained its effectiveness when linked to the gauze molecule. That is, the anti-microbial soluble hemostatic gauze has the same bacteria killing effect as the unlinked antibiotic.

TABLE 1
Gram-Positive Aerobic Bacillus Bacteria

| | Minimum Inhibited Concentration (mg/L) | |
|---|---|---|
| | | Anti-microbial gauze |
| Bacteria | Chlindamycin | with Chlindamycin |
| Staphylococcus Aureus | 1 | 2 |
| Staphylococcus Epidermidis | 0.5 | 0.5 |
| Streptococcus Faccalis | 1 | 1 |
| Streptococcus Gamma | 1 | 1 |
| Streptococcus Psittac | 1 | 1 |

TABLE 2
Gram-Negative Aerobic Bacillus Bacteria

| | Minimum Inhibited Concentration (mg/L) | |
|---|---|---|
| | | Anti-microbial gauze |
| Bacteria | Chlindamycin | with Chlindamycin |
| Pseudomonas Aeruginosa | 128 | 128 |
| Bacillus Pyocyaneus | 128 | 128 |
| Bacillus Coli Similis | 128 | 128 |
| Bacillus Coli Immobilis | 128 | 128 |
| Bacillus Canalis Capsulatus | 64 | 64 |
| Klebsiella Pneumoniae | 32 | 32 |
| Citrobacter Sp. | 64 | 64 |
| Serratia | 32 | 32 |

TABLE 3
Gram-Positive Anaerobic Bacillus Bacteria

| | Minimum Inhibited Concentration (mg/L) | |
|---|---|---|
| | | Anti-microbial gauze |
| Bacteria | Chlindamycin | With Chlindamycin |
| Bacteroides Fragilis | 0.5 | 0.5 |
| Bacteroides Vulgatus | 0.125 | 0.125 |
| Bacteroides Ovatus | 2 | 2 |
| Fusobacterium Nuleatum | 4 | 4 |

TABLE 4
Gram-Negative Anaerobic Bacillus Bacteria

| | Minimum Inhibited Concentration (mg/L) | |
|---|---|---|
| | | Anti-microbial gauze |
| Bacteria | Chlindamycin | with Chlindamycin |
| Clostridium Perfringens | 0.125 | 0.125 |
| Peptostrepococcus | 0.125 | 0.125 |
| Propionibacterium | 0.25 | 0.25 |
| Actinomyces | 0.125 | 0.125 |
| Lactobacillus Acidophilus | 32 | 32 |
| Lactobacillus Plantarum | 64 | 64 |
| Bifidobacterium Longum | 64 | 64 |
| Bifidobacterium Infantis | 64 | 64 |
| Bifidobacterium Adolescents | 16 | 16 |

Although we have disclosed certain present preferred embodiments of our soluble hemostatic gauze material and antibiotic containing soluble hemostatic gauze material it should be distinctly understood that our invention is not limited thereto but may be variously embodied within the scope of the following claims.

We claim:

1. A hemostatic gauze material comprising soluble cellulose molecules each having a carboxyl group and a pharmaceutically effective agent of the type containing an amino-group, the amino group attached to the carboxyl group of the cellulose molecules.

2. The hemostatic gauze material of claim 1 wherein the pharmaceutically effective agent is an antibiotic.

3. The hemostatic gauze material of claim 2 wherein the antibiotic is selected from the group consisting of actinomycin, amphotericin B, chlindamycin, erythromycin, lincomycin and neomycin.

4. The hemostatic gauze material of claim 1 wherein the pharmaceutically effective agent is a chemotherapy agent.

5. The hemostatic gauze material of claim 4 wherein the chemotherapy agent is selected form the group consisting of paclitaxel, bleomycin, mitomycine, gemcitabine, and fluorouracil.

6. The hemostatic gauze material of claim 1 wherein the cellulose molecules are formed by the steps of:
a. providing a cotton gauze;
b. immersing the cotton gauze in a solution of from 90% to 95% ethanol, methanol or acetone at room temperature;
c. exposing the cotton gauze to a solution of from 10% to 70% alkaline material at a temperature of from 20° C. to 50° C. for from 1 to 4 hours;
d. exposing the cotton gauze to a solution of from 20% to 80% of an acid or acid salt having a COOH group at a temperature of from 20° C. to 80° C. for from 1 to 6 hours to attach COOH groups to molecules in the cotton gauze;
e. washing the cotton gauze in ethanol, methanol or acetone at a temperature of from 10° C. to 60° C. for a sufficient time to neutralize the cotton gauze and remove residue salts from the gauze resulting in a soluble hemostatic gauze; and
f. drying the soluble hemostatic gauze.

7. The hemostatic gauze material of claim 1 wherein the gauze material has been sterilized.

8. The hemostatic gauze material of claim 1 also comprising a medicament selected from the group consisting of thrombin, tissue plasminogen activator, tissue plasminogen activator analogue, streptokinase, heparin, low molecular weight heparin, and pentasaccharide.

9. The hemostatic gauze material of claim 1 also comprising an adhesive strip on which the cellulose molecules are carried.

10. A method of making a soluble hemostatic gauze comprising:
a. providing a cotton gauze;
b. immersing the cotton gauze in a solution of from 90% to 95% ethanol, methanol or acetone at room temperature;
c. exposing the cotton gauze to a solution of from 10% to 70% alkaline material at a temperature of from 20° C. to 50° C. for from 1 to 4 hours;
d. exposing the cotton gauze to a solution of from 20% to 80% of an acid or acid salt having a COOH group at a temperature of from 20° C. to 80° C. for from 1 to 6 hours to attach COOH groups to molecules in the cotton gauze;
e. washing the cotton gauze in ethanol, methanol or acetone at a temperature of from 10° C. to 60° C. for a sufficient time to neutralize the cotton gauze and remove residue salts from the gauze resulting in a solible hemostatic gauze; and
f. drying the soluble hemostatic gauze.

11. The method of claim 10 wherein the washing step is comprised of:
a. washing the gauze in a first solution of ethanol, methanol or acetone at a temperature of from 10° C. to 60° C. for 10 to 60 minutes;
b. washing the gauze in a second solution of ethanol, methanol or acetone at a temperature of from 10° C. to 60° C. for 10 to 60 minutes; and
c. soaking the gauze in a third solution of ethanol, methanol or acetone at a temperature of from 10° C. to 60° C. for from 1 to 20 hours;

12. The method of claim 10 also comprising soaking the gauze in a solution of from 50% to 95% ethanol, methanol or acetone, 2% to 40% dimethyl sulfoxide and 2 mg/ml to 2 g/ml of a pharmaceutically effective agent containing an amino-group to link the amino group onto a COOH group attached to molecules in the cotton gauze.

13. The method of claim 12 wherein the pharmaceutically effective agent is an antibiotic.

14. The method of claim 13 wherein the antibiotic is selected from the group consisting of actinomycin, amphotericin B, chlindamycin, erythromycin, lincomycin and neomycin.

15. The method of claim 12 wherein the pharmaceutically effective agent is a chemotherapy agent.

16. The method of claim 16 wherein the chemotherapy agent is selected form the group consisting of paclitaxel, bleomycin, mitomycine, gemcitabine, and fluorouracil.

17. The method of claim 10 wherein the gauze is dried in an oven at a temperature of from 40° C. to 120° C.

18. The method of claim 10 also comprising cutting the soluble hemostatic gauze into desired shapes and sizes after the drying step.

19. The method of claim 10 also comprising sterilizing the soluble hemostatic gauze.

20. The method of claim 10 also comprising impregnating the gauze with a medicament selected from the group consisting of thrombin, tissue plasminogen activator, tissue plasminogen activator analogue, streptokinase, heparin, low molecular weight heparin, and pentasaccharide.

21. The method of claim 10 wherein the step of providing the cotton gauze comprises degreasing a cotton gauze in a manner to create a medical grade absorbent gauze.

22. The method of claim 10 wherein the acid or acid salt is acetic chloride and exposure occurs at a temperature of 55° C.
